(19) United States
(12) Patent Application Publication
(10) Pub. No.: US 2025/0144133 A1
(43) Pub. Date: May 8, 2025

(54) COLESTYRAMINE FOR TREATING WOUNDS, BACTERIAL INFECTIONS OF THE SKIN, AND INFLAMMATORY SKIN LESIONS

(71) Applicant: Dietrich SEIDEL, Feldafing (DE)

(72) Inventors: Dietrich SEIDEL, Feldafing (DE); Andreas WALTER, Landshut (DE)

(21) Appl. No.: 18/837,388

(22) PCT Filed: Feb. 9, 2023
(86) PCT No.: PCT/EP2023/053228

§ 371 Date: Aug. 9, 2024

(30) Foreign Application Priority Data
Feb. 9, 2022 (DE) 10 2022 103 062.8

(51) Int. Cl. A61K 31/785; A61K 45/06; A61L 26/00; A61P 17/02; A61P 31/02

(52) U.S. Cl. CPC A61K 31/785; A61K 45/06; A61L 26/0019; A61P 17/02; A61P 31/02

(57) ABSTRACT

The invention relates to an agent from the group of anion exchangers, preferably colestyramine, for the care and prophylaxis of wounds and injuries, in particular in the presence of local bacterial infections or critical colonizations, with the goal of biologically neutralizing bacterial toxins which disrupt the healing process during the healing of the wound. The application can be carried out by directly applying the product or in combination with conventional materials and modern simple wound care methods.

The present invention relates to the use of anion exchangers, in particular colestyramine, in wound care.

In the history of medicine since its inception (Hippocrates, 460-370 BC) and to this day, infections in general, but more particularly local, chronically infected wounds represent a serious medical problem, which also acquires a social dimension if one considers that in Germany alone about 2-3 million people are suffering from poorly healing or non-healing wounds. Remarkably, ca. 2% of the total health care budget is allocated annually in Germany for localised infections and poorly healing wounds, to the personal distress of affected patients, which can extend over many years.⁽¹⁾ In the USA, the expenditure for this is running at ca. 40 billion US dollars per year. With life expectancy rising at ca. 2 to 3 months per year, without measurable progress in treatment this problem will increase still further throughout the world.

Due to the typical colonization of gram-positive and gram-negative bacteria on the body surface, skin injuries and burns lead to critical colonization or wound infection. Here, Staphylococcus aureus, Escherichia coli, enterococci, MRSA, Proteus mirabilis, Klebsiella, ß-hemolytic streptococci and Pseudomonas aeruginosa play a decisive role.

Wound healing, in particular in the cutaneous region, is among the most complex processes of the human body.^{(1,2,3)}

Normally, wound healing takes place in definite phases, which can however be disrupted in many ways and progress thereof retarded, even leading to the development of chronic non-healing wounds. In the first phase, which is the exudative-inflammatory phase, the course of healing or impaired healing is initiated. If successful, this initial healing phase is followed by the proliferative phase and at a later stage the tissue remodeling phase.^{(1,2)}

The treatment of both deep-seated and surface, cutaneous wounds has changed markedly in the last two decades. Drying out wounds in order to prevent infection is outdated, as this leads to disruption of tissue repair and thus retarded wound healing. The aim of current modern wound therapy is rather to guarantee a physiological growth and healing environment in the region of the wound in order to thereby prevent pathological wound healing mechanisms and processes.^{(2,3,4)}

A first step in this was the introduction of hydroactive wound dressings for regulating the moisture balance in the wound region. In spite of the wide variety of products available for this purpose, from wound dressings and various drainage devices (endo sponges) to vacuum treatment, the superiority of individual products over others has not so far been proven in an evidence-based manner.^{(5,6)} As a result, there are still no binding guidelines for the standardization of modern wound management. This is to some extent explained by the fact that every wound treatment has to be individualized because of its complexity and has to be adapted to the particular patient.

A wound is defined as the loss of integrity of this organ due to exogenous or endogenous influences. Depending on the nature of the wound, local wound factors, systemic mediators and underlying causes (type of injury, burns, diabetes mellitus, arterial occlusive disease, varicose veins, genetically-determined anomalies, etc.), wound treatment may require an interdisciplinary therapeutic approach.

If the physiological process of wound healing is disturbed, particularly in the phase of exudation and incipient granulation, a chronic wound develops mostly on the basis of a prolonged inflammatory reaction. The physiological exudate of a wound is as a rule characterized by the activation of granulocytes, macrophages and fibroblasts, by significantly elevated concentrations of cytokines or chemokines (IL-1, IL-6, IL-8, TNF-α, inter alia), by proteases and elastases, as well as by an increase in growth factors (FDGF, PDGF), which together are necessary for the undisturbed proliferation of fibroblasts for wound covering.⁽³⁾

Microbial colonization of the wound leads persistently to a disruption of the physiological environment and furthermore influences the immunological status in loco. This has led to ever more intensive use of antiseptics (silver, iodine, etc.) and to an almost unrestricted administration of antibiotics in wound treatment. However, it was to be expected and is unmistakable that the uncontrolled use of such substances also leads to significant medical problems due to their cell and organ toxicity^{(7,8)} and, above all, to the development of multi-resistant bacterial strains (MRSA, ORSA or VRE). In addition, the necessarily high tissue levels of such substances often provoke undesired effects, and even severe allergies, cell toxicity and severe organ damage.^{(9,10)} Today, there is a general consensus that the efficacy of these substances can only be maintained if their medical use is sensitively controlled and not used on a long-term basis or only prophylactically in exceptional cases.

Of paramount pathophysiological importance in the development of chronically infected and poorly healing wounds, whether they are on the surface, deep-seated or in an encapsulated focus, are usually not the bacteria themselves, but rather their toxins, which are produced by killing the bacteria and can trigger adverse to severe local and systemic reactions. Depending on the immune status of the patient and the concentration of bacterial toxins, these not only have an adverse influence on wound healing itself, but also intensify the local inflammatory processes and can spread throughout the entire organism, which can often lead very rapidly to an organ failure, multi-organ failure and even septic shock. Even today, almost 50% of all affected patients die of this complication. (1) As little as 100 ng of a bacterial toxin can have considerable effects in humans, a few micrograms can cause death; these are the most toxic biological substances for humans at all.

In loco, the presence of bacterial toxins (such as lipopolysaccharides [LPS] of gram-negative bacteria and/or lipoteichoic acids [LTA] of gram-positive bacteria, mostly in combination) leads to the activation of the coagulation cascade, the impairment of fibrinolysis and the release of lysosomal enzymes and of peroxide radicals. This results in a massive shift in the mediator equilibrium in the wound exudate. In the complex inflammatory processes and repair processes, cytokines, immunomodulators and growth factors jointly as well as in their interaction have an important regulatory function. Elevated concentrations of bacterial toxins lead to the activation of monocytes, endothelial cells and neutrophil granulocytes. This results in increased expression of pro-inflammatory and immunomodulatory cytokines (IL-1β, TNF-α), further inflammatory mediators (histamine, kinins, nitric oxides) and an apoptosis disequilibrium in favor of the apoptosis-promoting members of the Bcl-2 group^{(2,3,12)} as well as profound shifts in the complement system and in the coagulation cascade. This explains the adverse action of bacterial toxins on the wound growth of fibroblasts, on the reduction of blood supply and hence on the negative balance of oxygen supply to the infected wound.^{(2,3)}

The colonization of a wound, in particular by problem pathogens, not only causes retarded wound healing due to the direct consequences such as abscess formation or tissue necrosis, but it in particular also delays the entire healing process and triggers a variety of complications through the release of LPS and LTA—inter alia as a consequence of the antibacterial and bactericidal therapy.

In order to achieve the therapeutic aim—to minimize the pathologically effective concentration of bacterial toxins in the wound region—there have been scarcely any effective therapeutic approaches in the strategy of modern wound management to date. Even a renaissance of old methods such as the use of silver ions, treatment with maggots, treatment with bees' honey, dressings with activated charcoal, etc., either individually or in combination with modern antibiotics, have remained without any demonstrable benefit in terms of reducing the pathologically effective concentration of bacterial toxins in the wound region.

Therefore, the problem underlying the present invention was to sustainably reduce the concentration of bacterial toxins locally by means of suitable materials or to render the bacterial toxins harmless without at the same time adversely influencing the physiological environment of the wound, i.e. the natural concentration ratios of the biological mediators in the wound exudate or disrupting their action through excessive fluid withdrawal and thus counteracting wound healing. It was also the task of the present invention to bind the bacterial toxins from the beginning of the infection or a critical bacterial colonization in a technically simple, effective and inexpensive manner and thereby render them harmless without permanently changing the physiological wound environment. Further, the aim was to minimize the undesired negative effects that are always to be expected in the context of a possible simultaneous antibiosis due to the increased release of bacterial toxins when the bacteria are killed.

Even though the agent used for solving these problems according to the present invention is mainly directed at the local treatment and prophylaxis of infection in skin wounds or susceptible skin areas (e.g. neurodermatitis, psoriasis, acne), this likewise applies to its use in any infected wound areas including abscesses in body cavities (peritonitis, pleural empyema, etc.), organs (e.g. liver abscess), in the bone tissue (e.g. osteomyelitis), or even for the treatment and prophylaxis of implant infections insofar as an introduction of the agent to the desired site of action is possible and medically appropriate.

WO 2012/123363 A1 describes approaches for using bacterial toxin-binding materials as components of wound dressings in order to achieve an improvement in wound healing. However, preference is given to wound dressing materials that are expensive to produce and high-priced hollow fiber materials with the greatest possible surface performance, to which chemically modified adsorption materials are attached in a specific manner. Such solutions have so far been neither economically nor clinically feasible, primarily for reasons of material costs.

Therefore, there is still a great need to improve the treatment of small and superficial wounds, as well as large or deep wounds, and to provide easy-to-use, effective and inexpensive means of doing so. Above all, there is still a need for means to treat people who have special and particularly serious injuries that cannot be adequately treated with the materials and drugs used to date or whose injuries are so serious that there is a high risk of death. Such injuries in particular include burns, especially extensive burns.

This problem is solved by providing an agent which is equipped with properties known from the technique of chromatography and which is suitable to act therapeutically even in the treatment of special skin wounds or injuries by adsorption and optionally elimination of bacterial toxins. This relates to the treatment of wounds and injuries of all kinds, both for the prevention and for the treatment of harmful effects of local bacterial infections. According to the invention, materials having the required characteristics have anion-exchanger groupings which are suitable for ionically/physically binding bacterial toxins, in particular lipopolysaccharides and lipoteichoic acids (LPS and LTA), and thereby biologically neutralizing them. The agent according to the present invention comprises one or more such anion exchangers as active ingredient.

Preferably, the anion exchangers used according to the present invention are polycationic molecules. These have polycation chains which are synthetic, semi-synthetic or natural, whereby mixed forms thereof are also suitable. The polycation chains are present in linear or branched form.

Preferred cationic groups of the polycation chains are tertiary amino groups and/or in particular quaternary ammonium groups. In particular, these are dialkylaminoalkyl or trialkylammoniumalkyl, dialkylaminoaryl or trialkylammoniumaryl, diarylaminoalkyl or triarylammoniumalkyl, diarylaminoaryl or triarylammoniumaryl moieties.

A preferred anion exchanger moiety includes trimethylbenzylammonium moieties. In the context of the present invention, the most preferred compound which can be used as an anion exchange moiety is colestyramine (poly(trimethylammoniomethylstyrene chloride-co-divinylbenzene)). Furthermore, the substance colesevelam, which is also already used pharmaceutically because of its anion-exchanger effect, is a suitable active agent in the context of the present invention.

In the context of the present invention, it is preferred that the anion exchanger, in particular the colestyramine, is present as a powder, fine powder or fine granules or is processed into a powder, fine powder or granules for incorporation into the agent. Advantageously, the colestyramine is used in a formulation that can be easily applied to the injury or wound. Any type of formulation in the form of a cream, gel or spray is particularly suitable for this purpose. Such formulations will be explained in more detail below.

The effect of colestyramine is based on ionic binding of the lipid portion of toxic bacterial components (LPS, LTA). It is an ion exchange resin in the form of small to very small particles. Products with different particle diameters down to nanometer range are commercially available and can be substituted according to the present invention.

Colestyramine is not soluble, not absorbable, it is not metabolized or stored in the body. As a basic substance, it is inexpensive to produce for the healthcare market.

According to the definition of its use, colestyramine is a medical product and not a drug. It has been used in gastroenterology for decades as an adsorbent of bile acids from the gastrointestinal tract. Undesirable effects resulting from the mode of action of colestyramine are not known, even at high doses and long-term use. In fact, the described adverse effects of oral use at high doses can be directly attributed to the reduction of bile salts.

In the context of the present invention, it was found that the application of the agent according to the present invention in the form of a preferably covering layer on the skin area to be treated has extremely positive effects on the healing of a wound or lesion. In the course of corresponding clinical studies, the use of the anion exchanger materials according to the present invention, and in particular colestyramine, not only led to a complete healing of chronic wounds, but also to a rapid healing of deep, acute wounds undisturbed by the negative effects of bacterial toxins. This accelerated healing tendency could be observed in both humans and animals in therapeutic trials, which is why both human and veterinary medical use is the subject of the present invention.

The chromatographically active anion exchanger material and in particular colestyramine, which is used in the treatment of often already infected, critically colonized wounds, prevents or reduces the negative effects of these toxins, as described above. The adsorptive binding and thus ultimate removal of endotoxins of gram-negative bacteria, lipopolysaccharides LPS, and also toxins of gram-positive bacteria, lipoteichoic acids LTA, from the wound environment facilitates a significant improvement in the pathophysiological conditions in the wound area, so that natural wound healing can proceed largely unhindered.

Further, it was found that the application of anion exchangers, in particular colestyramine, enables biological neutralization and removal of harmful bacterial toxins in an extremely effective manner, but that the physiological environment of the wound is hardly negatively affected, particularly with regard to the concentration ratios of biological mediators in the wound exudate.

Surprisingly, it was found in the context of the present invention that wound healing is very positively influenced by the clinical application of the agent according to the present invention, even if no anti-microbial substances are used. In contrast to the antiseptic treatment of infected wounds, which has been predominantly used up to now, the measures of the present invention can prevent the healing process from being negatively influenced by the increased release of bacterial toxins as a result of such antiseptic treatment and/or antibiosis. Insofar as it nevertheless appears appropriate to use antibiotics, e.g. systemically, the use of the agent according to the present invention can prevent a massive, local release of bacterial toxins from leading to the transfer of these toxins into the bloodstream and thus into the entire body of the patient, which triggers the known subsequent cascade, which progresses in a partially autoimmune-destructive manner via activation of mediators and in serious cases can even lead to septic manifestations up to septic shock and death of the patient.

A moist wound environment, which is preferably maintained when colestyramine is used according to the present invention due to a suitable formulation, enables a good contact between the wound secretion and the toxin binding sites and thus guarantees the highest effectiveness of the treatment.

As already mentioned above, besides superficial wounds also deep wounds, wound cavities and infections (e.g. post-operative) as well as abscess cavities and empyema may be suitable for treatment with the agent according to the present invention. In such wound conditions, the agent according to the present invention can be applied to or introduced into the wound, cavity or region. As with the treatment of superficial wounds, it is essential that the bacterial toxins are bound, do not spread in the wound environment and do not enter the bloodstream.

Patients with burns, in particular large-area burns, benefit in an unexpectedly positive way from the use of the agent according to the present invention.

Burn injuries are damage to the skin and the underlying tissue caused by heat, chemicals, electricity or radiation. The crucial difference between these and other acute or chronic wounds is that the protective barrier of the skin is damaged, sometimes over a large area. Serious consequences resulting therefrom include loss of fluids and electrolytes, infections and other serious complications.

Burns also differ from normal wound treatment when it comes to medical treatment. In contrast to normal wounds, which can often be treated with modern wound management, burns require special care.

In addition to compensating for fluid/electrolyte losses and intensive pain treatment (often through artificial coma), the urgent prevention of infections and serious inflammation is paramount. Inflammatory reactions are caused in particular by highly potent bacterial toxins (toxins), which severely disrupt the physiological balance which is required for normal wound healing. A veritable inflammatory tsunami descends on the tissue. In addition to the severe local inflammatory reactions, fulminant courses including sepsis and multi-organ failure can be the result. In addition, pronounced scarring caused by inflammation can lead to severe functional limitations later on and, not least, to considerable psychological problems.

Early intervention, including prophylactic intervention, can significantly reduce the risk of complications, improve long-term results and help patients to recover as fully and quickly as possible. However, such options are not yet sufficiently available.

The use of the agent according to the present invention for the treatment of burns, in particular burns with a severity of II or IIB and more severe, and in particular of patients with extensive burns (from 2-5 cm wound size or diameter, respectively, up to burns on entire limbs or body parts), is a new approach to solving the problems mentioned. The use of the agent according to the present invention, in particular colestyramine in the appropriate formulation, can make a decisive contribution to avoiding infections and serious tissue inflammation in the very special field of burn treatment. The reason for this is the highly effective binding and thus elimination of bacterial toxins, which has already been mentioned several times. Due to the large surface area of colestyramine in the form used according to the present invention, in particular as powder, fine powder or fine granules and in particular microspheres in the nanometer range, considerable quantities of toxins can be bound and neutralized.

According to the present invention, as a carrier substance of colestyramine in particular a water-based gel (hydrogel) can fulfill all the criteria for efficient therapeutic use. Accordingly, for the use of colestyramine according to the invention in the treatment of patients with severe and/or extensive burns, the use in a hydrogel formulation is a particularly preferred subject-matter of the present invention. By the combination with hydrogel, the active ingredient, preferably the colestyramine, is evenly distributed on the burn surface and fixed at the site of action over a longer period of time. In addition, a hydrogel counteracts the burn pain and supports the moisture balance of the tissue. The use of colestyramine gels according to the present invention has shown that even large-area burns heal within a very short time and that this healing can take place completely undisturbed by the harmful effects of ubiquitous bacteria, which pose such a great risk for the development of serious complications and ultimately septic conditions in the previous treatment regime for large-area burns. Surprisingly, additional local antibiotic treatment can be completely dispensed with when using the agent according to the present invention, which greatly supports wound healing.

Radiation damage, which in particular occurs in cancer patients after appropriate treatment, is also one of the burns which can be advantageously treated within the scope of the present invention and can be brought to rapid complete healing. The corresponding reports in the examples of the present application demonstrate the extraordinarily good effect, in particular also in the case of previously therapy-refractory radiation damage. Patients with such radiation damage therefore represent a further group of persons who unexpectedly benefit particularly strongly from the treatment according to the present invention with the colestyramine products of the present invention.

A further preferred embodiment of the present invention is in the field of poorly healing and previously at least partially refractory wounds of various kinds. Surprisingly, when the agent according to the present invention is used, it has also been shown that chronic wounds, in particular leg ulcers (ulcus cruris), which are a strong burden for many people, heal quickly and completely when the agent according to the present invention is used. This is a surprising effect and an unexpected technical effect of the present invention in that patients with such chronic lesions have usually already tried every available treatment method recommended to them by their doctors. However, in a large number of cases, despite the application of various wound regimes, including antibiotic treatment, final healing could not be achieved. Surprisingly, it has been shown that the present invention has also been able to achieve outstanding treatment successes in these cases and that wounds that have existed for years have finally healed.

The use of the agent according to the present invention for the treatment of patients with chronic and long-standing wounds (in particular wounds existing for several months or years) is therefore a further particularly preferred embodiment of the present invention.

Bedridden patients with more or less pronounced decubitus are another group of patients whose treatment has so far been very complex and sometimes unsuccessful. Unfortunately, existing wound treatment methods have been able to only achieve limited success in these cases too. The patients have to be bedded in a very complex manner and frequently turned. This is a considerable burden for hospital staff, but also for nursing staff in old people's homes and care facilities, who also look after long-term bedridden patients. In comparison with a “normal wound”, a decubitus is exposed to permanent stress and is often located in areas of the body where the environment is not conducive to healing. Decubitus patients are therefore exposed to considerable pain and negative health effects in addition to their underlying disease over a long period of time. Decubitus that cannot be treated are often the cause of patient death.

The agent according to the present invention can be applied very well to parts of the body affected by decubitus and can also remain on these areas, in particular if an appropriate covering is applied or any other protection against undesired removal of the agent is applied. Since experience has shown that healing begins very quickly after application of the agent according to the present invention, healing of the decubitus can also be observed within a period of days to a few weeks. This excellent effect of the agent according to the present invention must also be regarded as surprising, since ultimately inexpensive, easily available and uncomplicated materials are used to meet a long-standing need for medical application. A prophylactic application according to the present invention is also conceivable.

The active ingredient of the agent according to the present invention can be applied in pure form to the area to be treated, in particular if the anion exchanger is present as a powder or fine powder. In this embodiment, the agent according to the present invention consists only of the one or more anion exchangers; no carriers or excipients are present. In another embodiment, which is to be regarded as preferred for many applications, the active ingredient is present in a liquid to semi-solid or solid carrier base (e.g. in the form of suspension, gel, cream, lotion, ointment, paste, spray/stick spray, powder, etc.). This carrier base is a pharmaceutically or galenically acceptable base. Such base products for any dosage forms are known to the person skilled in the art. In the context of the invention formulations with hydrogels, i.e. in particular colestyramine in fine particulate form incorporated into a gel base, are particularly preferred.

The agent according to the present invention can be applied directly to the wound or inserted in advance into modern wound management materials (e.g. hydrofiber, foam dressing, alginate, etc.). Furthermore, the area to be treated can be protected after application of the agent according to the present invention by covering it with a conventional wound dressing and, if necessary, fixing it in place. This can be a compress, a plaster or any other dressing material. This dressing material can be air-permeable, but should reliably hold the applied agent in place. Such a dressing can also protect against soiling, mechanical damage or even drying out or unwanted oozing or the like. Suitable materials are known to the person skilled in the art. In a particularly preferred embodiment of the present invention, the agent according to the present invention, preferably the colestyramine gel, is applied to a material that is impermeable to the gel. This can be, for example, a film to which a layer of the gel is applied, so that this product can be packaged ready for use and preferably also offered in sterile form. The simple application of such a ready-to-use product facilitates the application and thus also the discomfort for the patient, especially in the case of large-area burns.

The concentration of the active ingredient according to the present invention (preferably colestyramine) in the carrier base results from clinical experience in the respective form of application. In preferred but non-limiting embodiments of the present invention, the agent includes the active ingredient in a carrier base at a concentration of 0.5 to 20% by weight, although other concentrations may also be indicated depending on the form of application. Concentrations of 1 to 15% by weight, 2 to 10% by weight and in particular 3 to 8% by weight are more preferred, especially for gels, creams, lotions and ointments. As already mentioned above, the active ingredient can also be used pure, resulting in a 100% active ingredient concentration. When used as a powder or paste with corresponding carriers, which then also fall under the definition of a suitable carrier base, high concentrations of active ingredient may also be present, for example up to 95%, 90%, 80%, 75%, 50%, in each case based on weight, or the above concentrations of 0.5 to 20% by weight may also be suitable for these dosage forms.

Finally, it is also conceivable to formulate the composition in the form of a shower gel, shampoo or other body cleansing or body care product, in particular for infection prophylaxis. For this purpose, suitable substances, such as washing-active substances, are included in the formulation of the agent according to the present invention.

Suitable gelling agents or gelling agent mixtures known to the skilled person can be used for the application as a gel. The gel base may, for example, include substances in particular from the group consisting of alginates, hyaluronic acid and hyaluronates, polyacrylic acid such as Carbopol® polymers, propylene glycol, carboxymethyl cellulose, tragacanth gum, silicon dioxide, gelatine, methyl cellulose, poloxamers or povidone. It may also be desirable to use a commercially available gel base. In particular, gel bases that form at least a temporary layer on the wound or skin are suitable. Other suitable gel bases are hydrogels consisting of water and a water-insoluble polymer, or those based on colloid/sol.

In a further preferred embodiment of the invention, the agent according to the present invention is placed in a moisture- or liquid-permeable container with suitable dimensions, which is suitable for application to the area to be treated. The container can be a bag, a cushion or a pouch/bag, a cushion fixed to a plaster or similar products. In this embodiment of the present invention, the agent according to the invention is not applied directly to the wound or lesion, rather the active ingredient in the container is in an exchange with the wound/wound secretion due to the moisture permeability and can also bind bacterial toxins with high effectiveness in this embodiment.

If desired, the agent according to the present invention may include one or more further active ingredients. Such active ingredients may preferably be selected from the group comprising analgesics, anti-inflammatory agents, antiseptics, antibiotics, antimycotics, antiallergic agents and antiviral substances. Mixtures of these substances and other supporting substances can also be added to the agent according to the present invention. By way of example and as being preferred according to the present invention, the following can be mentioned: Silver ions, zinc oxide, povidone-iodine, activated charcoal, ibuprofen, acetylsalicylic acid, diclofenac, dexpanthenol and mixtures of two or more of these substances. The formulation of an agent according to the present invention also corresponds to the above possibilities in this case. Depending on the further active ingredient and its properties, the agent can therefore be used pure, i.e. undiluted and without carriers or excipients. Alternatively, the other dosage forms can be used using suitable carrier bases and additives as explained above. In particular, suitable preservatives may also be added.

Due to its good binding properties for LPS and LTA, colestyramine, which is preferably used as an active ingredient according to the present invention, can be used surprisingly well in combination with antibiotics or other bactericidal agents without the associated increased release of toxins causing undesirable effects. Thus, a therapy with antibiotics (systemic or local) or other bactericidal agents can be supported by the agent according to the present invention and thus wound healing can be significantly improved.

The agent according to the present invention is directed to the prevention of infection of acute wounds and the accelerated healing of chronic, superficial and deep wounds with critical bacterial colonization or infection. Corresponding treatment options exist in particular for dermatitis in general, neurodermatitis, efflorescences of the skin, burns, folliculitis, boils, carbuncles, acne, decubitus, subcutaneous and deep-seated infections, abscesses and empyema, etc.

Within the scope of the invention, the treatment can be carried out both acutely and for infection prophylaxis, the latter in particular for burns, decubitus, ulcus cruris and neurodermatitis. The agent according to the present invention also offers good treatment success for open legs (ulcus cruris), decubitus and other chronic wounds, in particular those caused by diabetes mellitus, arterial occlusive disease and varicose veins, as well as for their prevention as soon as the first lesions appear.

The invention represents a completely new and cost-effective concept for wound treatment, which has already proven to be extremely effective when applied to selected patients.

In addition to the agent according to the present invention, combinations with other anion exchangers as the active ingredient, as well as with other agents and products known in wound management, are also a subject-matter of the present invention.

In addition to the above-mentioned medical applications, the agent according to the present invention is also of particular interest for cosmetic applications. While clinically relevant acne often requires the treatment of open and already infected wounds, mild courses of acne in both adolescents and adults are more of a cosmetic problem. In the early phase of the development of pimples, pustules or blackheads, a duct of a hair follicle becomes blocked for various reasons and a retention cyst forms. If this cyst inflames, a purulent pustule forms. As a rule, such a pustule opens by itself or can easily be opened manually. Once the pustule has been opened, treatment is indicated in order to promote rapid healing without scarring or to prevent the infection from spreading to other areas of the skin. However, treatment of the closed pustule may also be indicated, e.g. to prevent additional bacteria from entering when the pustule is opened. Furthermore, a retention cyst that is not yet infected can also be treated with the aid of the agent according to the invention.

The agent according to the present invention is particularly suitable for these cosmetic applications, as it can be applied easily and without complications and even a generous application beyond the actual site of the skin impurity is completely unproblematic.

These advantages in application apply to the agent in powder form as well as to an agent in semi-solid or liquid form (e.g. as a spray or tincture). Rapid drying of the formulation on open skin and preferably the presence of film-forming excipients or carriers enable the agent according to the present invention to remain at the site of action for a sufficiently long time without visual impairment.

A further subject-matter of the present invention is therefore the non-therapeutic, cosmetic application of the agent according to the present invention for skin blemishes of all kinds, in particular mild to moderately severe blemished skin or acne, or an agent for use in the aforementioned cases.

The invention is illustrated by the following examples and treatment reports.

### EXAMPLES

### Example 1: In Vitro Adsorption and Elimination of LPS and LTA from a Simulated Wound Exudate in the Presence of Cytokines and a Growth Factor

Comparison of Colestyramine with Another Anion Exchanger

The simulated wound exudate consisted of human albumin (2 weight percent), calcium chloride (0.02 M), sodium chloride (0.4 M) and trimethylamine (0.08 M), dissolved in distilled water. The pH value was adjusted to pH 7.5.

The following bacterial toxins, human cytokines and a representative growth factor were added to the simulated wound exudate:
A. Lipopolysaccharides (LPS, 2 EU/mL, E. coli 055: B5 endotoxin; BioWhitaker)
B. Lipoteichoic acids (LTA, 1 μg/mL, Streptococcus pyogenes; Sigma Aldrich)
C. Recombinant human tumor necrosis factor alpha (rh TNF-α, 10 pg/ml; Biomol)
D. Recombinant human interleukin-6 (rh IL-6, 50 pg/mL; Biomol)
E. Recombinant human platelet-derived growth factorA (rhPDGF A; 50 ng/ml; Biomol)

An aliquot (5 mL) of simulated wound exudate or wound exudate mixed with bacterial toxins, human cytokines and growth factor was spiked with 100 mg of each of the adsorbents listed below, incubated for 4 hours at 37° C. and then centrifuged off at 1000 g.

### Adsorbents Used:

1. Colestyramine (Quantalan, Bristol Meyer Squibb)
2. DEAE-modified polyamide hollow fiber (corresponding to DE 10258944 A1; B. Braun Melsungen A G)

The substances (A-E) added to the simulated wound exudate have the following molecular weights: A and B) common lipid A subunit˜2000 Dalton; C) ˜50000 Dalton; D) ˜25000 Dalton; E) ˜30000 Dalton.

### Determination Methods:

LPS was determined using the chromogenic kinetic limulus amebocyte lysate test (LAL-QCL, Cambrex).

The detection of LTA was carried out indirectly by the aid of a whole blood stimulation test. For this purpose, freshly collected heparinized whole blood from a healthy donor was mixed with the untreated or treated prepared wound exudate in a ratio of 6/1 (v/v) and incubated for 24 hours at room temperature. After centrifugation (3000 g for 10 min.) and collection of the corresponding plasma fraction, the interleukin-6 (IL-6) biosynthesis rate stimulated by the LTA was determined. Recombinant human interleukin-6 (IL-6) and rh PDGF-A were quantified using a corresponding ELISA test (Biomol GmbH).

The concentration of recombinant human TNF-α was determined using an EIA kit (Biomol GmbH).

### Results:

After incubation with the prepared wound exudate and removal of the used adsorbents by centrifugation, the percentage depletion (elimination rate) of the added components (A-E, i.e. bacterial toxins, cytokines and growth factor) was determined in the respective supernatant.

The following elimination rates [%] were found:

Adsorber

| | 1 | 2 |
|---|---|---|
| LPS | 95 | 85 |
| LTA | 95 | 90 |
| TNF | 10 | 70 |
| IL-6 | 10 | 80 |
| PDGF-A | 5 | 80 |

The example shows that colestyramine is as effective as materials known for LPS/LTA adsorption. However, other substances such as cytokines and growth factors are not bound in significant quantities, so that the physiological mediators of wound healing remain virtually unaffected.

### Example 2: Therapy Reports from the Clinical Application

Patient 1 presents with condition after open abdominal treatment with healing and remaining scar of approx. 8×12 cm.

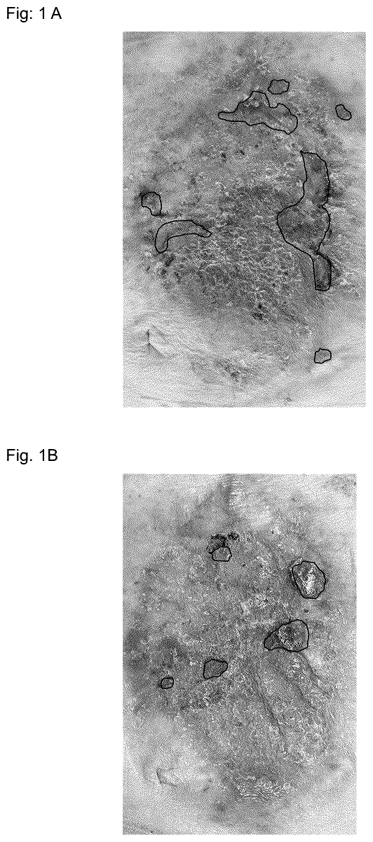

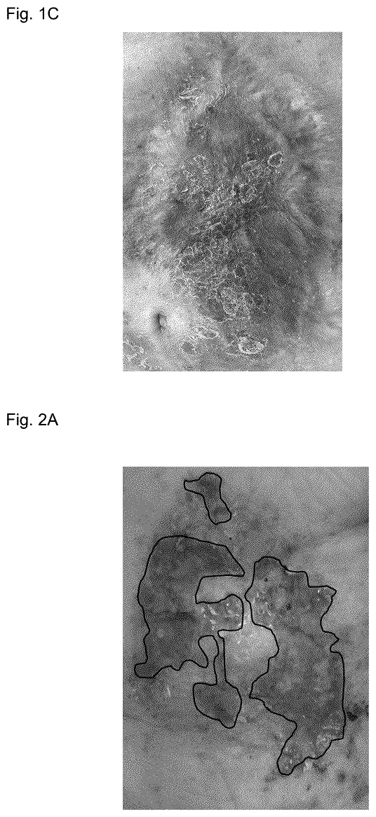

After the start of tumor therapy, severe ulcerations occur again (FIG. 1A, see black outline). Treatment was then started with a gel according to the present invention (6% colestyramine in a gel base (aqua, propylene glycol, panthenol, sodium carbomer, sodium hydroxide, phenoxyethanol, ethylhexylglycerol, sodium hyaluronate)), which was applied in a thin layer (approx. 1 mm) to the wound.

After 2 weeks, there is a clear reduction in the signs of inflammation and a visible size reduction of the wound area (FIG. 1B, see black outline).

After a further 4 weeks of treatment, the wound was completely closed—despite continued tumor therapy (FIG. 1C).

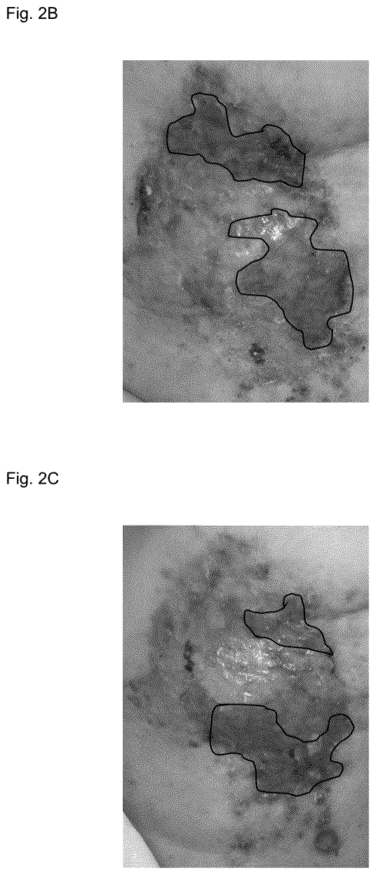

Patient 2 suffers from a locally advanced breast carcinoma. A pronounced dermatitis developed under radiation (FIG. 2A, see black outline). Although she has been undergoing specialized wound treatment for months, the patient still complains of severe burning pain.

Treatment was then started with gel (6% colestyramine in a gel base (aqua, propylene glycol, panthenol, sodium carbomer, sodium hydroxide, phenoxyethanol, ethylhexylglycerol, sodium hyaluronate)), which was applied in a thin layer (approx. 1 mm) to the wound. After 2 weeks, there was a clear reduction in the signs of inflammation and symptoms of discomfort (FIG. 2B).

After a further 4 weeks of treatment with the colestyramine gel according to the present invention, there was a further reduction in the signs of inflammation (FIG. 2C) and the patient was almost symptom-free.

Patient 3, female age 51 years, suffered a burn on the forearm and hand with redness, skin injury and blistering when putting wood in an open fireplace on an iron part, stage burn II with redness on a skin area of approx. 2×10 cm on the forearm and approx. 2×4 cm on the hand, blistering over 70-80% of the wound area. The treatment was started on the same day, the gel according to the present invention (6% colestyramine in a gel base (aqua, propylene glycol, panthenol, sodium carbomer, sodium hydroxide, phenoxyethanol, ethylhexylglycerol, sodium hyaluronate) was applied by the patient in a thin layer to the entire injured skin area. The application was repeated twice daily. The patient was pain-free after just 2 days and the burn wounds had healed completely after 5 days. (Oral report from the patient who applied the colestyramine gel herself).

Patient 4, male, had been treated inpatient in an accident hospital for several weeks due to an accident. He was bedridden and had developed a large decubitus, which was then treated with the gel according to the present invention (6% cholestyramine, aqua, propylene glycol, panthenol, sodium carbomer, sodium hydroxide, phenoxyethanol, ethylhexylglycerol, sodium hyaluronate). Within a few days, the decubitus began to heal and after 3 weeks of treatment, the decubitus was already considerably reduced in size to approx. 50% of its original size. After a further two weeks, the decubitus had healed completely.

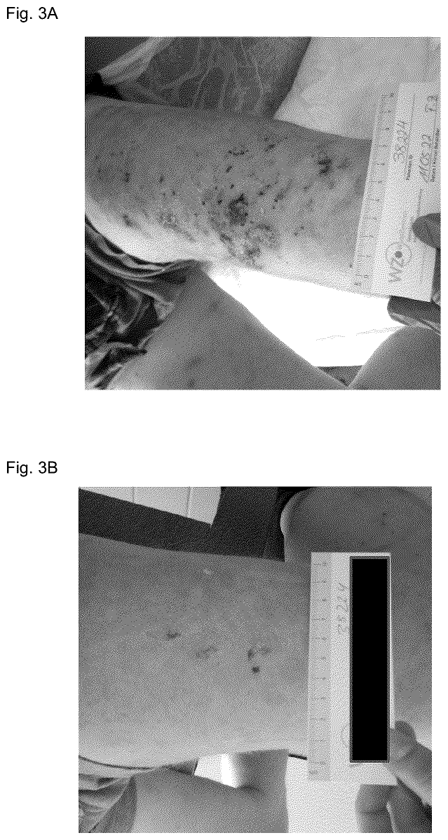

Patient 5, male, 56 years old, suffers from prurigo nodularis with chronic ulcerative dermatitis. The status after 8 months of treatment with modern wound management is shown in FIG. 3A. After 3.5 months of treatment with cholestyramine in hydrogel (6% cholestyramine, aqua, propylene glycol, panthenol, sodium carbomer, sodium hydroxide, phenoxyethanol, ethylhexylglycerol, sodium hyaluronate), the wound was almost completely closed (FIG. 3B).

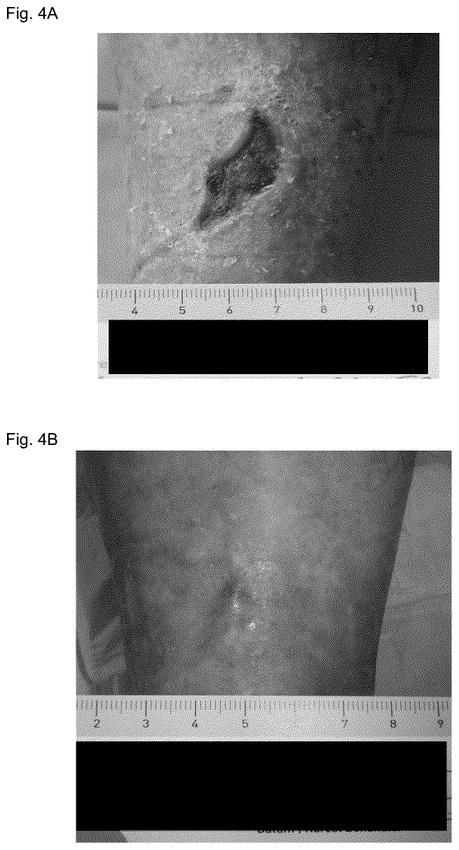

Patient 6, female, 80 years old, suffered from a post-traumatic right-sided ulcus crusis with venous insufficiency. After split-thickness skin grafting with partial graft rejection and protracted wound treatment with modern wound management, the treatment status was extremely unsatisfactory, as is shown in FIG. 4A.

After 8 weeks of treatment with the colestyramine gel according to the present invention (6% cholestyramine, aqua, propylene glycol, panthenol, sodium carbomer, sodium hydroxide, phenoxyethanol, ethylhexylglycerol, sodium hyaluronate), complete wound closure was observed (FIG. 4B).

### LISTING OF REFERENCES

### 1. J. Heinlin et al.

Wundheilung—Therapeutische Interventionen

Hautarzt 2010, 61:611-628

### 2. Y Winkler

Endotoxin-Bindung, bakteriozide Wirksamkeit und Zytotoxizität silberhaltiger Wundauflagen

Inauguraldissertation, Ernst-Moritz-Arndt-Universität Greifswald, 2010

### 3. M. Schäffer, H.-D. Becker

Immunregulation der Wundheilung

Chirurg 1999, 70:897-908

### 4. H. Lippert

Wundatlas. Kompendium der komplexen Wundbehandlung

Georg Thieme Verlag K G, Stuttgart/New York, 2006

### 5. M. Voshege et al.

Was ist evidenzbasiert in der Behandlung chronischer Wunden?

Gefäßchir 2003, 8:269-276,

### 6. P. K. Baier et al.

Subcutaneous Redon drains do not reduce the incidence of surgical site infections after laparotomy. A randomized controlled trial on 200 patients Int J Colorectal Dis 2010, 25:639-643

### 7. S. Trambe et al.

In vitro evaluation of the risk of developing bacterial resistance to antiseptics

Atimicrob Chemother 2001, 47:589-598

### 8. P. Appelgren et al.

A prospective study of infections in burn patients

Burns 2002, 28:39-46

### 9. G. Müller et al.

Biocompatibility index of antiseptic agents by parallel assessment of antimicrobial activity and cellular cytotoxicity

J Antimicrob Chemother 2008, 61:1281-1287

### 10. W Lineaweaver et al.

Topical antimicrobial toxicity

Arch Surg 1985, 120:267-270

### 11. R. Stone

Search for sepsis drugs goes on despite past failures

Science 1994, 264:365-367

### 12. Z. Metzger et al.

The effect of bacterial endotoxin on the early tensile strength of healing surgical wounds

J Endod 2002, 28:30-33

### 13. Xin Liu et al.

In vivo wound healing and antibacterial performances of electrospun nanofibre membranes

J Biomed Mater Res A. 2010, 94:499-508

1. An agent for use in the treatment of wounds or lesions of the skin and/or underlying tissues, in particular for the prevention and/or treatment of harmful effects of local bacterial infections or critical colonizations in the wound or lesion area, characterized in that it comprises as active ingredient an anion exchanger which is capable of binding bacterial toxins and thereby biologically neutralizing them.

2. The agent for use according to claim 1, characterized in that the wounds or lesions of the skin and/or underlying tissues are a burn of at least grade 2, in particular 2 or 2b.

3. The agent for use according to claim 1, characterized in that the wounds or lesions of the skin and/or underlying tissues are a chronic wound, in particular an ulcus cruris.

4. The agent for use according to claim 1, characterized in that the wounds or lesions of the skin and/or underlying tissues are a decubitus, in particular a decubitus of at least grade 2 or grade 3.

5. The agent for use according to claim 1, characterized in that it includes colestyramine or/and colesevelam, preferably colestyramine, as active ingredient.

6. The agent for use according to claim 1, characterized in that the active ingredient is presented as a raw substance or in a galenically acceptable base in liquid to semi-solid or solid form, in particular as a suspension, gel, cream, lotion, ointment, paste, spray/adhesive spray or powder.

7. The agent for use according to claim 1, characterized in that it is applied or placed directly on the infected or inflammable region or is placed in a moisture- and/or liquid-permeable container, for example a bag, cushion or pocket, or is applied to a moisture- and/or liquid-impermeable film or other support.

8. The agent for use according to claim 1, characterized in that it is integrated in a conventional wound dressing.

9. The agent for use according to claim 1, characterized in that the active ingredient is incorporated into a matrix forming an adhesive layer on the wound or skin.

10. The agent for use according to claim 1, characterized in that, in addition to the anion exchanger, it includes one or more further active ingredients in combination.

11. The agent for use according to claim 10, characterized in that in the combination one or more further active ingredients are selected from the group of antibiotics, analgesics, antiphlogistics, antiseptics, antimycotics, antiallergics, antiviral substances, silver ions, zinc oxide, povidone-iodine, activated charcoal, ibuprofen, acetylsalicylic acid, diclofenac, local anaesthetics, dexpanthenol and mixtures thereof.

12. The agent for use according to claim 1, characterized in that the wound or lesion to be treated is a superficial or deep wound, an open, infected acute or chronic wound, a subcutaneous infection, a deep-seated infected region, optionally also after implantation of a foreign object, an efflorescence of the skin, a burn, a folliculitis, a boil, acne, a dermatitis, a neurodermatitis or a decubitus.

13. The agent for use according to claim 1, characterized in that it is also used for prophylactic application in the case of a particular genetic disposition, as well as in the case of a disposition to neurodermatitis, burns or decubitus, in order to prevent critical colonization or bacterial infection, respectively.

14. Non-therapeutic use of an agent containing an anion exchanger, in particular colestyramine, as active ingredient for cosmetic purposes, wherein the agent is preferably characterized as in claim 5.

15. Non-therapeutic use of an agent according to claim 14 for the treatment or prophylaxis of mild to moderate acne, pimples, pustules and blackheads.

16. A method for the treatment of wounds or lesions of the skin and/or underlying tissues, in particular for the prevention and/or treatment of harmful effects in local bacterial infections or critical colonizations in the wound or lesion area, characterized in that it comprises as active ingredient an anion exchanger which is capable of binding bacterial toxins and thereby biologically neutralizing them.

17. The use according to claim 16, characterized in that the agent is characterized as active ingredient.
